SEQUENCE LISTING (12 sequences)

SEQ ID NO: 1 - protein, 165 aa, synthetic construct
MASISEKMVEALNRQINAEIYSAYLYLSMASYFDSIGLKGFSNWMRVQWQEELMHAMKMFDFVSERGGRVKLYAVEEPPSEWDSPLAAFEHVYEHEVNVTKRIHELVEMAMQEKDFATYNFLQWYVAEQVEEEASALDIVEKLRLIGEDKRALLFLDKELSLRQF

SEQ ID NO: 2 - protein, 166 aa, synthetic construct
MASISEKMVEALNRQINAEIYSAYLYLSMASYFDSIGLKGFSNWMRVQWQEELMHAMKMFDFVSERGGRVKLYAVEEPPSEWDSPLAAFEHVYEHEVNVTKRIHELVEMAMQEKDHATYNFLQWYVAEQVEEEASALDIVEKLRLIGEDKRALLFLDKELSLRQTS

SEQ ID NO: 3 - protein, 166 aa, synthetic construct
MASISEKMVEALNRQINAEIYSAYLYLSMASYFDSIGLKGFSNWMRVQWQEELMHAMKMFDFVSKRGGRVKLYAVEEPPSEWDSPLAAFEHVYEHEVNVTKRIHELVEMAMQEKDHATYNFLQWYVAKQVKEEASALAIVEKLRLIGEDKRALLFLDKELSLRQTS

SEQ ID NO: 4 - protein, 166 aa, synthetic construct
MASISEKMVEALNRQINAEIYSAYLYLSMASYFDSIGLKGFSNWMRVQWQEELMHAMKMFDFVSQRGGRVKLYAVEEPPSEWDSPLAAFEHVYEHEVNVTKRIHELVEMAMQEKDHATYNFLQWYVAEQVEEEASALAIVEKLRLIGEDKRALLFLDKELSLRQTS

SEQ ID NO: 5 - protein, 166 aa, synthetic construct
MASISEKMVEALNRQINAEIYSAYLYLSMASYFDSICLKGFSNWMRVQWQEELMHAMKMFDFVSERCGRVKLYAVEEPPSEWDSPLAAFEHVYEHEVNVTKRIHELVEMAMQEKDHCTYNFLQWYVAEQVEEEASALDIVEKLRLIGEDKRCLLFLDKELSLRQTS

SEQ ID NO: 6 - protein, 166 aa, synthetic construct
MASISEKMVEALNRQINAEIYSAYLYLSMASYFDSICLKGFSNWMRVQWQEELMHAMKMFDFVSKRCGRVKLYAVEEPPSEWDSPLAAFEHVYEHEVNVTKRIHELVEMAMQEKDHCTYNFLQWYVAKQVKEEASALAIVEKLRLIGEDKRCLLFLDKELSLRQTS

SEQ ID NO: 7 - protein, 166 aa, synthetic construct; identical to SEQ ID NO: 3

SEQ ID NO: 8 - protein, 166 aa, synthetic construct
MASISEKMVEALNRQINAEIYSAYLYLSMASYFDSICLKGFSNWMRVQWQEECMHAMKMFDFVSERCGRVKLYAVEEPPSEWDSPLAAFEHVYEHEVNVTKRIHELVEMAMQEKDHCTYNFLQWYVAEQVEEEASALDIVEKLRLIGEDKRCLLFLDKELSLRQTS

SEQ ID NO: 9 - protein, 166 aa, synthetic construct
MASISEKMVEALNRQINAEIYSAYLYLSMASYFDSICLKGFSNWMRVQWQEECMHAMKMFDFVSKRCGRVKLYAVEEPPSEWDSPLAAFEHVYEHEVNVTKRIHELVEMAMQEKDHCTYNFLQWYVAKQVKEEASALAIVEKLRLIGEDKRCLLFLDKELSLRQTS

SEQ ID NO: 10 - protein, 166 aa, synthetic construct
MASISEKMVEALNRQINAEIYSAYLYLSMASYFDSICLKGFSNWMRVQWQEECMHAMKMFDFVSQRCGRVKLYAVEEPPSEWDSPLAAFEHVYEHEVNVTKRIHELVEMAMQEKDHCTYNFLQWYVAEQVEEEASALAIVEKLRLIGEDKRCLLFLDKELSLRQTS

SEQ ID NO: 11 - protein, 12 aa, synthetic construct
YHWYGYTPQNVI

SEQ ID NO: 12 - protein, 6 aa, synthetic construct
LARLLT

(19) United States
(12) Patent Application Publication
(10) Pub. No.: US 2026/0053945 A1
(43) Pub. Date: Feb. 26, 2026

(54) GASTROINTESTINAL TRACT STABILIZED PROTEIN DELIVERY USING DISULFIDE TES SYSTEM

(71) Applicant: National University of Singapore, Singapore (SG)

(72) Inventors: Chester Lee DRUM, Singapore (SG); Girish VALLERINTEAVIDE MAVELLI, Singapore (SG); Samira SADEGHI, Singapore (SG)

(21) Appl. No.: 19/105,336

(22) PCT Filed: Aug. 24, 2023
(86) PCT No.: PCT/SG2023/050584

§ 371 Date: Feb. 20, 2025

(30) Foreign Application Priority Data
Aug. 25, 2022 (SG) 10202250830B

(51) Int. Cl. A61K 47/69; A61K 9/00; A61K 9/51; C07K 14/47

(52) U.S. Cl. CPC A61K 47/6931; A61K 9/0053; A61K 9/5169; C07K 14/47; C07K 2319/00

(57) ABSTRACT

The present invention relates to an engineered disulphide-linked ferritin assembly comprising at least one modified ferritin subunit, wherein the at least one modified ferritin subunit comprises the amino acid sequence set forth in SEQ ID NO: 1, and comprises i) a F116H substitution, and zero or more amino acid substitutions at one or more positions selected from the group comprising E65, E128, E131, and D138 of SEQ ID NO: 1, and ii) a Cys substitution at two or more positions selected from the group comprising G37, L53, R66, G67, A74, A117 and A152 of SEQ ID NO: 1. The present invention also relates to its uses and manufacture.

### CROSS-REFERENCE TO RELATED APPLICATIONS

The present application is a filing under 35 U.S.C. 371 as the National Stage of International Application No. PCT/SG2023/050584, filed Aug. 24, 2023, entitled “GASTROINTESTINAL TRACT STABILIZED PROTEIN DELIVERY USING DISULFIDE TES SYSTEM,” which claims priority to Singapore Application No. 10202250830B filed with the Intellectual Property Office of Singapore on Aug. 25, 2022, both of which are incorporated herein by reference in their entirety for all purposes.

### INCORPORATION BY REFERENCE OF MATERIAL IN XML FILE

This application incorporates by reference the Sequence Listing contained in the following XML file being submitted concurrently herewith:

File name: 4373-20500 SP105840USZ Sequence Listing, created on Aug. 22, 2023; and having a file size of 12.5 KB. The information in the Sequence Listing is incorporated herein in its entirety for all purposes.

### FIELD OF THE INVENTION

The present invention relates to thermostable ferritin assemblies, in particular, engineered disulphide-linked ferritin assemblies having enhanced stability to acidic pH and human digestive enzymes, thereby also stabilizing an encapsulated payload, and increased intestinal permeability compared to a reference ferritin assembly without added disulphide links. The present invention also relates to an in vitro method of forming an engineered disulphide-linked ferritin assembly, a method of delivering an engineered disulphide-linked ferritin assembly into a cell; and a kit to encapsulate a molecule within said engineered disulphide-linked ferritin assembly.

### BACKGROUND OF THE INVENTION

Oral is the most preferred and convenient mode among all drug administration routes and is the dominant mode of drug administration for common medicines. Oral delivery offers the best patient compliance as drugs are self-administered with flexible dosages following the patients' needs [Sadeghi, S., et al., ACS nano (2022); Majoul, I., et al., FEBS Letters 401:104-108 (1997); Okumura, M., et al., The FEBS Journal 279:2283-2295 (2012)]. In addition, the gastrointestinal (GI) tract mucosa and epithelial cells exhibit a large surface area of 30-40 m² and thus the opportunity for high mass transfer and absorption. Goblet cells, endocrine cells, Paneth cells, and specialized M cells on the GI tract may likewise perform specialized roles in drug adsorption offering unique characteristics for oral drug development [Deshpande, S., et al., Nature communications 8:1-8 (2017); Zhang, M., et al., International Journal of Biological Macromolecules 138:636-647 (2019)]. Although a standard for small molecule administration routes, the field of nanomedicine has yet to find a material base for consistent oral drug delivery due to the often delicate superstructures required of biocompatible polymers and lipids in the context of high acidity and unfavorable physicochemical properties of the GI tract. Ultimately, nanoparticle delivery technologies remain with poor bioavailability and a need for alternative materials which are stable throughout the GI tract [Sadeghi, S., et al., ACS nano (2022); Martien, R., et al., Nanotechnology 19: 045101 (2008); Wu, W., et al., Food Hydrocolloids 80:78-87 (2018)].

Proteins and peptides are widely used in clinical care with only <5% administered orally, mostly due to the instability at pH variations and degradative enzymes in the stomach and proximal small intestine [Ezpeleta, I., et al., International Journal of Pharmaceutics 191:25-32 (1999)]. Nanoparticle systems have improved the oral delivery of small molecule drugs, but relatively little progress has been made using functionally active proteins or enzymes, owing to the incompatibility of organic emulsions used for the synthesis of these systems and the tertiary structures of proteins required for activity. Thus, the combination of denaturation and low encapsulation efficiency results in an ongoing challenge [Bies, C., et al., Advanced drug delivery reviews 56:425-435 (2004); Minko, T. Advanced drug delivery reviews 56:491-509 (2004); Penalva, R., et al., Food hydrocolloids 44:399-406 (2015)]. Highly stabilized nanoparticles are a promising approach toward biocompatibility, biodegradability, non-immunogenicity, resistance to enzymatic degradation, and amenability to genetic modifications for surface engineering [Shapira, A., et al., Nanomedicine 6:119-126 (2010); Penalva, R., et al., Journal of Agricultural and Food Chemistry 63:5603-5611 (2015); Umamaheshwari, R., et al., Aaps Pharmscitech 5:60-68 (2004)]. One such approach is the self-assembled and monodisperse protein cage, ferritin, engineered to have enhanced circulation half-life and low toxicity [Umamaheshwari, R., et al., Aaps Pharmscitech 5:60-68 (2004); Irache, J. M. and Gonzalez-Navarro, C. J. Future Medicine 12:1209-1211 (2017); Patel, A R., et al., Journal of Agricultural and Food Chemistry 58:12497-12503 (2010)]. Currently, however, gastric pH causes these protein cages to disassemble, exposing the therapeutic protein cargo and leading to activity loss and degradation by digestive enzymes [Sadeghi, S., et al., ACS nano (2022); Hong, S., et al., Pharmaceutics 12:604 (2020); Kianfar, E. Journal of Nanobiotechnology 19:1-32 (2021); Vallerinteavide Mavelli, G. et al., Pharmaceutics 13: 1790 (2021)]. Further, these cages have to traverse through the thick mucus layer on the GI tract to reach the lumen, limiting their application as an oral delivery vehicle [Wu, W., et al., Food Hydrocolloids 80:78-87 (2018); Estrada, L. H. and Champion, J., Biomaterials Science 3:787-799 (2015)].

There is a need to create an oral delivery vehicle having improved stability within the gastrointestinal tract.

### SUMMARY OF THE INVENTION

Thermostable exoshells (tES) are engineered proteinaceous nanoparticles used for the rapid encapsulation of therapeutic proteins/enzymes, whereby the nanoplatform protects the payload from proteases and other denaturants. Given the significance of oral delivery as the preferred model for drug administration, the inventors structurally improved the stability of tES through multiple inter-subunit disulfide linkages that were initially absent in the parent molecule. The disulfide-linked tES, as compared to tES, significantly stabilized the activity of encapsulated horseradish peroxidase (HRP) at acidic pH and against the primary human digestive enzymes, pepsin, and trypsin. Furthermore, the disulfide-linked tES (DS-tES) exhibited significant intestinal permeability as evaluated using Caco2 cells. An in vivo bioluminescence assay showed that encapsulated Renilla luciferase (rluc) was ˜3 times more stable in mice compared to the free enzyme. The faeces of DS-tES-treated mice had ˜100 times more active enzyme in comparison to the control (free enzyme) after 24 h of oral administration, demonstrating strong intestinal stability. Taken together, the in vitro and in vivo results confirm the potential of DS-tES for intraluminal and systemic oral drug delivery applications.

The inventors developed a novel protein cage ‘tES’ by engineering the thermostable Archaeoglobus fulgidus ferritin, which are 12 nm in diameter with an 8 nm aqueous cavity, and a volume that can theoretically accommodate protein(s) of molecular volume ˜270 nm³. The shells contain four 4.5 nm surface pores, which allow small molecule substrates to permeate while blocking the interaction of macromolecules such as antibodies and proteases [He, J., et al., Journal of Controlled Release 311:288-300 (2019)]. The advantages of tES are: (1) simple and rapid encapsulation of protein cargo using charge complementation and mild pH titrations, (2) prevention of aggregation, aiding in proper tertiary structures and maintenance of protein activity, and (3) highly stabilized and protective environment for internalized proteins against denaturation and proteolysis, making it an ideal vector for enzyme delivery. Additionally, tES has been shown to function as an artificial chaperone to fold proteins in vitro [Khoshnejad, M., et al., Journal of Controlled Release 282:13-24 (2018)], stabilize labile proteins from freezing and drying stresses during lyophilization [Sadeghi, S., et al., Nature Communications 12:1-15 (2021)], and serve as a multilayered catalytic center for bioorthogonal catalysis to treat solid tumors [Yin, S. et al., Particuology 64:65-84 (2022)]

Here, the inventors describe the structural refinement of tES by incorporating multiple inter-subunit disulfide linkages to create a disulfide-linked tES with practicable advantages in oral delivery applications. By engineering natural covalent crosslinking using molecularly precise disulfide linkages, i.e. DS-tES, the inventors were able to protect encapsulated enzymes against the adverse GI environment when compared to tES and/or enzyme alone. Furthermore, in an in vitro Caco2 assay DS-tES exhibited permeabilization through intestinal epithelial cells, confirming enhanced absorption. Finally, rluc encapsulated within DS-tES displayed clear protein stabilization throughout all sections of the GI tract via the clear presence of bioluminescence in faeces following oral administration in mice.

A description of exemplary, non-limiting, embodiments of the invention follows.

In a first aspect of the invention there is provided an engineered disulphide-linked ferritin assembly comprising at least one modified ferritin subunit, wherein the at least one modified ferritin subunit comprises the amino acid sequence set forth in SEQ ID NO: 1, and comprises;
i) a F116H substitution, and zero or more amino acid substitutions at one or more positions selected from the group comprising E65, E128, E131, and D138 of SEQ ID NO: 1, and
ii) a Cys substitution at two or more positions selected from the group comprising G37, L53, R66, G67, A74, A117 and A152 of SEQ ID NO: 1.
   The Cys substitutions form inter-subunit disulphide linkages that confer the following advantages:
1. Covalently locked shell
2. Disulfide bridges do not affect the overall morphology of tES
3. Stability to extreme acidic conditions (pH 4.0-pH 5.0)
4. Stability against proteolytic enzymes (eg. Pepsin, trypsin)

The free thiols on the tES surface have mucoadhesive properties and intestinal permeability.

The disulphide-linked ferritin assembly of the invention is able to encapsulate different molecules, proteins and enzymes, and stabilize them, thus providing a wide range of potential applications for drug delivery. A potential application of this disulphide-linked ferritin assembly is as a gastrointestinal stabilized oral delivery vehicle, because it can protect encapsulated molecules from adverse gastrointestinal conditions, including pepsin degradation and low pH conditions.

In some embodiments, the disulphide-linked ferritin assembly possesses a net positive interior charge, or a net neutral interior charge.

In certain embodiments, the engineered ferritin assembly that encapsulates a polypeptide can be delivered into a cell (e.g., in vivo delivery) in a composition or formulation comprising the engineered ferritin assembly and one or more pharmaceutically acceptable carriers or excipients. Suitable pharmaceutical carriers typically will contain inert ingredients that do not interact with the agent or nucleic acid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's lactate and the like. Formulations can also include small amounts of substances that enhance the effectiveness of the active ingredient (e.g., emulsifying agents, solubilizing agents, pH buffering agents, wetting agents). Methods of encapsulation compositions (such as in a coating of hard gelatin or cyclodextran) are known in the art.

In some embodiments, the modified ferritin subunit comprises an amino acid substitution at any one or more positions selected from E65, E128, E131, and D138 corresponding to the positions in the peptide sequence set forth in SEQ ID NO: 1.

In some embodiments, the amino acid substitution comprises;
5i) substitutions E65K, E128K, E131K and D138A, which cause the assembly to have a net positive interior charge; or
ii) substitutions E65Q and D138A, which cause the assembly to have a net neutral interior charge.

In some embodiments, prior to Cys substitution the modified ferritin subunit comprises an amino acid sequence selected from the group comprising SEQ ID NO: 2, SEQ ID NO: 3, and SEQ ID NO: 4.

In some embodiments, the modified ferritin subunit comprises cysteine residues at positions selected from the group comprising A152C-G67C-A117C-G37C and A152C-L53C-G67C-A117C-G37C corresponding to the positions in the peptide sequence set forth in SEQ ID NO: 1.

In some embodiments, the modified ferritin subunit comprises an amino acid sequence selected from the group comprising SEQ ID NO: 5, SEQ ID NO: 6, SEQ ID NO: 7, SEQ ID NO: 8, SEQ ID NO: 9 and SEQ ID NO: 10.

In some embodiments, a bioorthogonal catalytic centre, polypeptide, or nucleic acid, or any combination thereof, is encapsulated within the disulphide-linked ferritin assembly.

In a second aspect, the invention provides an in vitro method of forming an engineered disulphide-linked ferritin assembly of the first aspect, the method comprising adjusting the pH from an acidic pH to basic pH of a sample comprising at least one modified ferritin subunit of the first aspect, wherein the assembly is stable in the gastrointestinal tract and capable of oral drug delivery.

In some embodiments, the method further comprises a bioorthogonal catalytic centre, polypeptide or a nucleic acid, or any combination thereof, in the sample whereby said bioorthogonal catalytic centre, polypeptide or nucleic acid, or any combination thereof, is encapsulated by the engineered ferritin assembly at basic pH of the sample.

In some embodiments, the acidic pH in the sample is at least about 4.0 and/or the basic pH in the sample is about 8.0. In some embodiments, the acidic pH in the sample is at least 4.0 and/or the basic pH in the sample is 8.0.

In a third aspect, the invention provides a method of delivering an engineered disulphide-linked ferritin assembly into a cell, comprising contacting the cell with a disulphide-linked ferritin assembly of the first aspect.

In some embodiments, the disulphide-linked ferritin assembly further comprises a cell-targeting moiety, e.g., on the surface of the engineered ferritin assembly. In certain embodiments, the cell targeting moiety binds to a molecule (e.g., a receptor or a ligand) on the target, perhaps diseased, cell. In some embodiments the target cell is a cancer cell. As the disulphide-linked ferritin assembly is highly advantageous for oral therapeutic applications, the surface modification of the said with these peptides can help in greater targeting to tumours on the gastro-intestinal area. Examples of molecules bound by a cell targeting moiety include, e.g., EGFR and cell adhesion molecules (e.g., integrins and organ level selectivity such as the enhanced permeability and retention (EPR) effect).

As those skilled in the art would appreciate, a variety of molecules (e.g., peptides, receptors, ligands, and fragments thereof) can be used as a targeting moiety to target and bind to a molecule on a diseased cell. In certain embodiments, the diseased cell is a cancer cell and the cell-targeting moiety is a peptide comprising the amino acid sequence set forth in SEQ ID NO: 11 (YHWYGYTPQNVI) or SEQ ID NO: 12 (LARLLT).

In some embodiments, the targeting moiety is cross-linked to the disulphide-linked ferritin assembly surface. Methods of crosslinking molecules (e.g., peptides) with other molecules are known in the art. For example, as described herein, crosslinking agents, e.g., sulfo-SMCC, can be used to attach a targeting moiety onto the surface of an engineered disulphide-linked ferritin assembly.

In some embodiments, the engineered ferritin assembly that encapsulates a polypeptide can be delivered into a cell (e.g., in vivo delivery) in a composition or formulation comprising the engineered ferritin assembly and one or more pharmaceutically acceptable carriers or excipients. Suitable pharmaceutical carriers typically will contain inert ingredients that do not interact with the agent or nucleic acid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's lactate and the like. Formulations can also include small amounts of substances that enhance the effectiveness of the active ingredient (e.g., emulsifying agents, solubilizing agents, pH buffering agents, wetting agents). Methods of encapsulation compositions (such as in a coating of hard gelatin or cyclodextran) are known in the art.

In a fourth aspect, the invention provides an isolated plasmid or vector polynucleic acid comprising a sequence that encodes a modified ferritin subunit, wherein the modified ferritin subunit is as defined in the first aspect.

In a fifth aspect, the invention provides a composition comprising at least one engineered disulphide-linked ferritin assembly for use in the prophylaxis or treatment of disease in a subject.

In some embodiments, the at least one engineered disulphide-linked ferritin assembly has encapsulated:
i) an enzyme; or
ii) an enzyme and one or more additional therapeutic agents.

In some embodiments, the composition is capable of drug delivery by the oral route.

In a sixth aspect, the invention provides a method of treatment or prophylaxis comprising administering to a subject in need of such treatment or prophylaxis an efficacious amount of a composition of the fifth aspect.

In some embodiments, the composition is administered sequentially or simultaneously with another therapeutic agent.

When administered in a combination therapy, the engineered ferritin assembly that encapsulates a polypeptide can be administered before, after or concurrently with the other therapy. When co-administered simultaneously (e.g., concurrently), the engineered ferritin assembly that encapsulates a polypeptide and other therapy can be in separate formulations or the same formulation. Alternatively, the engineered ferritin assembly that encapsulates a polypeptide and other therapy can be administered sequentially, as separate compositions, within an appropriate time frame as determined by a skilled clinician (e.g., a time sufficient to allow an overlap of the pharmaceutical effects of the therapies). In certain embodiments, the engineered ferritin assembly described herein is comprised in a composition or combination for use in the treatment of disease in a subject. In certain embodiments the disease is cancer.

In some embodiments, the composition comprises a bioorthogonal catalytic centre encapsulated within the engineered disulphide-linked ferritin assembly and the other therapeutic agent is a substrate for said catalytic centre.

In a seventh aspect, the invention provides a use of a composition of the fifth aspect for the manufacture of a medicament for the treatment of a disease in a subject.

In some embodiments, the disease is a cancer.

In an eighth aspect the invention provides a kit to encapsulate a bioorthogonal catalytic centre, polypeptide, or nucleic acid, or any combination thereof, within an engineered disulphide-linked ferritin assembly, the kit comprising at least one modified ferritin subunit according to the first aspect.

The invention also provides a kit to encapsulate a bioorthogonal catalytic centre, polypeptide or polynucleic acid, or any combination thereof, within an engineered disulphide-linked ferritin assembly, the kit comprising the plasmid or vector polynucleic acid of the fourth aspect.

### BRIEF DESCRIPTION OF THE FIGURES

FIGS. 1a-1c show (FIG. 1a) a schematic diagram of the sequential engineering of DS-tES. Non-reducing SDS-PAGE of the selected mutants, whereby lane 1 has mutant A152C; lane 2 has mutant A52C-L53C; lane 3 has mutant A152C-G67C; lane 4 has mutant A152C-L53C-G67C; lane 5 has mutant A152C-L53C-G67C-A117C-G37C (DS-tES)). (FIG. 1b) Schematic representation of DS-tES construct with five point mutations G37C-L53C-G67C-A117C-A152C and restriction sites. (FIG. 1c) PyMOL representation of DS-tES with all the point mutations (black balls represent the cysteine residues).

FIGS. 2a-2d show photographs of non-reducing SDS-PAGE for designed Cysteine residue substitutions. Lane 1 tES, lane 2 tES R151C, lane 3 tES A152C, lane 4 tES A152C-L53C, lane 5 tES A152C-A74C, lane 6 tES A152C-G76C, lane 7 tES A152C-R66C, lane 8 tES A152C-L53C-R66C, lane 9 tES A152C-A74C-R66C, lane 10 tES A152C-G67C, lane 11 tES A152C-L53C-G67C, lane 12 tES A152C-A74C-G67C, lane 13 tES A152C-G67C-A117C-G37C, lane 14 tES A152C-L53C-G67C-A117C-G37C. M=size marker.

FIGS. 3a-3c show (FIG. 3a) SDS-PAGE analysis of DS-tES and tES in the absence or presence of tris(2-carboxyethyl) phosphine (TCEP), a reducing agent that breaks disulfide bonds. (FIG. 3b) Size-exclusion chromatography of tES and DS-tES with similar elution profiles. (FIG. 3c) Hydrodynamic diameter measurements of tES and DS-tES using dynamic light scattering studies.

FIGS. 4a-4g show photographs of non-reducing SDS-PAGE to increase disulfide formation in 9, 10, and 11 constructs.

FIGS. 5a-5d show (FIG. 5a) size-exclusion chromatography of tES, A152C-G67C-A117C-G37C mutated tES, and DS-tES at pH 4.0. (FIG. 5b) Pepsin assay under different pH conditions. DS-tES protected the encapsulated HRP from the simulated gastric condition. (FIG. 5c) Trypsin assay, tES and DS-tES protect the HRP against trypsin enzyme. (FIG. 5d) Permeability assay in Caco2 monolayers shows that both tES and DS-tES can permeabilize through intestinal epithelium. FIG. 5b-FIG. 5d: Data are shown as mean±SEM, n=3. ***P<0.001.)

FIG. 6 shows an Ellman's assay standard curve.

FIGS. 7a-7c show (FIG. 7a) a schematic representation of oral administration of DS-tES-rluc/rluc followed by IP injection of viviRen substrate. (FIG. 7b) Bioluminescence emitted from mice administered with DS-tES-rluc/rluc in 3 and 24 h. (FIG. 7c) Luciferase activity measurement from mice fecal matter for up to 1 week. (FIG. 7b and FIG. 7c) Data are shown as mean±SEM, n=4. ***P<0.001, **P<0.01, *P<0.05.)

FIG. 8 shows that a permeability assay in Caco2 monolayers confirms that both tES and DS-tES can permeabilize through intestinal epithelium. (Data are shown as mean±SEM, n=3. ***P<0.001. and **P<0.01)

FIGS. 9a-9b show bioluminescence emitted from mice administered with tES-rluc/rluc in 3 h.

### DETAILED DESCRIPTION OF THE INVENTION

Bibliographic references mentioned in the present specification are for convenience listed in the form of a list of references and added at the end of the examples. The whole content of such bibliographic references is herein incorporated by reference. Any discussion about prior art is not an admission that the prior art is part of the common general knowledge in the field of the invention.

### Definitions

Unless defined otherwise, all technical and scientific terms used herein have the same meaning as is commonly understood by one of skill in the art to which the invention belongs. Certain terms employed in the specification, examples and appended claims are collected here for convenience.

As used herein, “a” or “an” may mean one or more than one unless indicated to the contrary or otherwise evident from the context.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

As used herein, the term “disulphide link” or “disulphide bridge” refers to inter-subunit covalent disulphide bonds formed between cysteine residues. The cysteine residues may be modified residues located in the tES dimerization intersection, at positions such as arginine 151(R151), alanine 152(A152), leucine 53(L53), alanine 74(A74), glycine 76(G76), arginine 66(R66), glycine 67(G67), glutamine 50(Q50), glycine 37(G37), and alanine 117(A117).

As used herein, “ferritin assembly” is used synonymously with ferritin, a known structure in the art, that comprises 24 subunits (ferritin subunits), each subunit having a defined size (e.g., ˜20 kDa for AfFtn) and structural motif (e.g., a four-helix bundle structural motif for AfFtn). The native ferritin assembly has a defined external diameter (e.g., 12 nm for AfFtn) and a defined internal cage (e.g., 8 nm for AfFtn).

As used herein, a ferritin subunit refers, as noted above, to one of 24 subunits that form the ferritin assembly. Subunits of the ferritin assembly have a defined structure, comprising a four-helix bundle motif. In AfFtn, each subunit is ˜20 kDa and contains a four-helix bundle structural motif. An AfFtn wildtype subunit has the amino acid sequence set forth in SEQ ID NO: 1 (Table 1).

TABLE 1
Amino acid sequence of AfFtn subunits

| | |
|---|---|
| SEQ ID NO: 1 | MASISEKMVE ALNRQINAEI |
| AfFtn wildtype subunit | YSAYLYLSMA SYFDSIGLKG |
| (unstructured C-terminal | FSNWMRVQWQ EELMHAMKMF |
| sequence underlined) | DFVSERGGRV KLYAVEEPPS |
| | EWDSPLAAFE HVYEHEVNVT |
| | KRIHELVEMA MQEKDFATYN |
| | FLQWYVAEQV EEEASALDIV |
| | EKLRLIGEDK RALLFLDKEL SLRQFTPPAE |
| | EEK |
| SEQ ID NO: 2 | MASISEKMVE ALNRQINAEI |
| AfFtn subunit with | YSAYLYLSMA SYFDSIGLKG |
| truncation of unstructured | FSNWMRVQWQ EELMHAMKMF |
| C-terminus | DFVSERGGRV KLYAVEEPPS |
| and F116H mutation | EWDSPLAAFE HVYEHEVNVT |
| | KRIHELVEMA MQEKDHATYN |
| | FLQWYVAEQV EEEASALDIV |
| | EKLRLIGEDK RALLFLDKEL SLRQTS |
| SEQ ID NO: 3 | MASISEKMVE ALNRQINAEI |
| AfFtn net positive variant | YSAYLYLSMA SYFDSIGLKG |
| subunit with truncation of | FSNWMRVQWQ EELMHAMKMF |
| unstructured C-terminus | DFVSKRGGRV KLYAVEEPPS |
| and F116H mutation | EWDSPLAAFE HVYEHEVNVT |
| | KRIHELVEMA MQEKDHATYN |
| | FLQWYVAKQV KEEASALAIV |
| | EKLRLIGEDK RALLFLDKEL SLRQTS |
| SEQ ID NO: 4 | MASISEKMVE ALNRQINAEI |
| AfFtn net neutral variant | YSAYLYLSMA SYFDSIGLKG |
| subunit with truncation of | FSNWMRVQWQ EELMHAMKMF |
| unstructured C-terminus | DFVSQRGGRV KLYAVEEPPS |
| and F116H mutation | EWDSPLAAFE HVYEHEVNVT |
| | KRIHELVEMA MQEKDHATYN |
| | FLQWYVAEQV EEEASALAIV |
| | EKLRLIGEDK RALLFLDKEL SLRQTS |
| SEQ ID NO: 5 | MASISEKMVE ALNRQINAEI |
| AfFtn subunit with | YSAYLYLSMA SYFDSICLKG |
| truncation of unstructured | FSNWMRVQWQ EELMHAMKMF |
| C-terminus, F116H | DFVSERCGRV KLYAVEEPPS |
| mutation and A152C- | EWDSPLAAFE HVYEHEVNVT |
| G67C-A117C-G37C | KRIHELVEMA MQEKDHCTYN |
| | FLQWYVAEQV EEEASALDIV |
| | EKLRLIGEDK RCLLFLDKEL SLRQTS |
| SEQ ID NO: 6 | MASISEKMVE ALNRQINAEI |
| AfFtn net positive variant | YSAYLYLSMA SYFDSICLKG |
| subunit with truncation of | FSNWMRVQWQ EELMHAMKMF |
| unstructured C-terminus, | DFVSKRCGRV KLYAVEEPPS |
| F116H mutation and | EWDSPLAAFE HVYEHEVNVT |
| A152C-G67C-A117C- | KRIHELVEMA MQEKDHCTYN |
| G37C | FLQWYVAKQV KEEASALAIV |
| | EKLRLIGEDK RCLLFLDKEL SLRQTS |
| SEQ ID NO: 7 | MASISEKMVE ALNRQINAEI |
| AfFtn net neutral variant | YSAYLYLSMA SYFDSIGLKG |
| subunit with truncation of | FSNWMRVQWQ EELMHAMKMF |
| unstructured C-terminus, | DFVSKRGGRV KLYAVEEPPS |
| F116H mutation and | EWDSPLAAFE HVYEHEVNVT |
| A152C-G67C-A117C- | KRIHELVEMA MQEKDHATYN |
| G37C | FLQWYVAKQV KEEASALAIV |
| | EKLRLIGEDK RALLFLDKEL SLRQTS |
| SEQ ID NO: 8 | MASISEKMVE ALNRQINAEI |
| AfFtn subunit with | YSAYLYLSMA SYFDSICLKG |
| truncation of unstructured | FSNWMRVQWQ EECMHAMKMF |
| C-terminus, F116H | DFVSERCGRV KLYAVEEPPS |
| mutation and A152C- | EWDSPLAAFE HVYEHEVNVT |
| L53C-G67C-A117C-G37C | KRIHELVEMA MQEKDHCTYN |
| | FLQWYVAEQV EEEASALDIV |
| | EKLRLIGEDK RCLLFLDKEL SLRQTS |
| SEQ ID NO: 9 | MASISEKMVE ALNRQINAEI |
| AfFtn net positive variant | YSAYLYLSMA SYFDSICLKG |
| subunit with truncation of | FSNWMRVQWQ EECMHAMKMF |
| unstructured C-terminus, | DFVSKRCGRV KLYAVEEPPS |
| F116H mutation and | EWDSPLAAFE HVYEHEVNVT |
| A152C-L53C-G67C- | KRIHELVEMA MQEKDHCTYN |
| A117C-G37C | FLQWYVAKQV KEEASALAIV |
| | EKLRLIGEDK RCLLFLDKEL SLRQTS |
| SEQ ID NO: 10 | MASISEKMVE ALNRQINAEI |
| AfFtn net neutral variant | YSAYLYLSMA SYFDSICLKG |
| subunit with truncation of | FSNWMRVQWQ EECMHAMKMF |
| unstructured C-terminus, | DFVSQRCGRV KLYAVEEPPS |
| F116H mutation and | EWDSPLAAFE HVYEHEVNVT |
| A152C-L53C-G67C- | KRIHELVEMA MQEKDHCTYN |
| A117C-G37C | FLQWYVAEQV EEEASALAIV |
| | EKLRLIGEDK RCLLFLDKEL SLRQTS |

As used herein, a “modified ferritin subunit” refers to a ferritin subunit that lacks an unstructured carboxy-terminal sequence that a wildtype ferritin subunit possesses. That is, the modified ferritin subunit comprises a deletion of amino acids 165-173 of SEQ ID NO: 1. Table 1 summarizes the sequences described herein. For instance, the subunit sequence is truncated at position 164 of SEQ ID NO: 1 and comprises a F116H substitution, as set forth in SEQ ID NO: 2 (Table 1).

As used herein, an “engineered ferritin assembly” refers to a ferritin assembly formed with at least one modified ferritin subunit, as described herein with reference to Deshpande, S., et al., Nat. Commun. 8, 1-8 (2017). The engineered ferritin assembly is sometimes referred to as a “nanoshell” or “nanocage” or “nanoencapsulator”, “NE” or “thermostable exoshell” or “tES”. An example of a modified ferritin subunit comprising residues 1-164 is set forth in SEQ ID NO: 2. As those of skill in the art would recognize, residues can be present beyond the last residue in the modified ferritin subunit that results from, e.g., cloning sites, that do not impact the engineered ferritin assembly function. For example, as shown in Table 1, AfFtn subunit with a truncation of the unstructured C-terminus comprise the residues “TS” that resulted from a cloning site (restriction site) in the cloning plasmid. Removal of the unstructured carboxy-terminal sequence not only frees up space inside the engineered ferritin assembly but also imparts stability to the assembly in low salt concentrations (e.g. below 30 mM NaCl).

The modified ferritin subunit can comprise one or more further modifications (e.g., amino acid substitutions) to its amino acid sequence. As described herein, at least one ferritin subunit can be modified to contribute to a net charge (e.g., positive, negative, or neutral) of the interior surface of the ferritin assembly. Methods of determining which residues to modify within a ferritin subunit to achieve a ferritin assembly possessing a net positive, negative, or neutral interior charge are known in the art, as described herein. An engineered ferritin assembly formed from native AfFtn ferritin subunit truncations possesses a net positive charge; an example of which is set forth in SEQ ID NO: 3. An engineered ferritin assembly formed from native AfFtn ferritin subunit truncations possesses a net neutral charge; an example of which is set forth in SEQ ID NO: 4. Mutants with residues substituted with Cysteine at various positions, are shown as SEQ ID NOs: 5 to 10.

As used herein, “protein” and “polypeptide” are used interchangeably to denote a polymer of at least two amino acids covalently linked by an amide bond, regardless of length or post-translational modification (e.g., glycosylation or phosphorylation). The term “protein” encompasses a naturally-occurring as well as artificial (e.g., engineered or variant) full-length protein as well as a functional fragment of the protein.

The term “functional fragment” refers to a portion of a protein that retains some or all of the activity or function (e.g., biological activity or function, such as enzymatic activity) of the full-length protein, such as, e.g., the ability to bind and/or interact with or modulate another protein or nucleic acid. The functional fragment can be any size, provided that the fragment retains, e.g., the ability to bind and interact with another protein or nucleic acid.

Preferably, the thermostable ferritin assembly modified subunit comprises the amino acid sequence set forth in SEQ ID NOs: 5 to 10, or a variant thereof which retains the ability to assemble. The choice of subunit sequence may depend on the desired acid resistance and permeability.

As would be well appreciated by a person skilled in the art, the biological function of a polypeptide/protein is closely linked to its folding and structure. Therefore, as used herein, the term “reducing structural loss” refers to minimising any changes in the structure/conformation of a polypeptide, minimising degradation of its intended structure, increasing its resistance to factors that may affect its intended structure or a combination thereof, which results in its improved ability to maintain the intended structure over a time period. Similarly, as used herein, the term “reducing functional loss” refers to minimising any changes in the intended function of a polypeptide, minimising the effectiveness of its intended function, increasing its resistance to factors that may affect its intended function or a combination thereof, which results in its improved ability to maintain the intended function over a time period.

As used herein, the term “substrate” refers to any molecule that may be encapsulated by the tES of the present disclosure. Preferably, the substrate is a labile substrate. Preferably, the labile substrate is a labile molecule. As used herein, a “labile molecule” refers to any molecule that is sensitive to any change(s) in its external environment such as but not limited to temperature, concentration, ionic charge and pH, wherein said change(s) in its external environment may affect its intended structure, conformation and/or function.

In some embodiments, the labile molecule may be a polypeptide, a nucleic acid or a macromolecule. In some embodiments, the labile molecule may have a therapeutic application. In some embodiments, the labile molecule is a macromolecule. In some embodiments, the labile molecule may be a macromolecule comprising an enzyme for conversion of a prodrug. In some embodiments, the labile molecule is horseradish peroxidase (HRP).

As used herein, the term “polynucleic acid” refers to a polymer comprising multiple nucleotide monomers (e.g., ribonucleotide monomers or deoxyribonucleotide monomers). “Polynucleic acid” includes, for example, genomic DNA, cDNA, RNA, and DNA-RNA hybrid molecules. Polynucleic acid molecules can be naturally occurring, recombinant, or synthetic. In addition, polynucleic acid molecules can be single-stranded, double-stranded or triple-stranded. In certain embodiments, polynucleic acid molecules can be modified. In the case of a double-stranded polymer, “polynucleic acid” can refer to either or both strands of the molecule.

For in vivo delivery, the reconstituted composition comprising engineered ferritin assembly that encapsulates a polypeptide can be delivered to a subject in need thereof by a variety of routes of administration including, for example, oral, dietary, topical, transdermal, or parenteral (e.g., intra-arterial, intravenous, intramuscular, subcutaneous injection, intradermal injection) routes of administration. Administration can be local or systemic. The actual dose of a therapeutic polypeptide encapsulated by an engineered ferritin assembly and treatment regimen can be determined by a skilled physician, taking into account the nature of the condition being treated, and patient characteristics.

Unless otherwise indicated or otherwise evident from the context and understanding of one of ordinary skill in the art, values that are expressed as ranges can assume any specific value or subrange within the stated ranges in various embodiments, to the tenth of the unit of the lower limit of the range, unless the context clearly dictates otherwise. “About” in reference to a numerical value generally refers to a range of values that fall within ±10%, in some embodiments ±5%, in some embodiments ±1%, in some embodiments+0.5% of the value unless otherwise stated or otherwise evident from the context. In any embodiment in which a numerical value is prefaced by “about”, an embodiment in which the exact value is recited is provided. Where an embodiment in which a numerical value is not prefaced by “about” is provided, an embodiment in which the value is prefaced by “about” is also provided. Where a range is preceded by “about”, embodiments are provided in which “about” applies to the lower limit and to the upper limit of the range or to either the lower or the upper limit, unless the context clearly dictates otherwise. Where a phrase such as “at least”, “up to”, “no more than”, or similar phrases, precedes a series of numbers, it is to be understood that the phrase applies to each number in the list in various embodiments (it being understood that, depending on the context, 100% of a value, e.g., a value expressed as a percentage, may be an upper limit), unless the context clearly dictates otherwise. For example, “at least 1, 2, or 3” should be understood to mean “at least 1, at least 2, or at least 3” in various embodiments. It will also be understood that any and all reasonable lower limits and upper limits are expressly contemplated.

Unless otherwise indicated, all technical and scientific terms used herein have the same meaning as commonly understood to one of ordinary skill in the art to which the invention belongs.

Having now generally described the invention, the same will be more readily understood through reference to the following examples which are provided by way of illustration, and are not intended to be limiting of the present invention.

### EXAMPLES

### Example 1

### Preparation of Disulfide Linked tES (DS-tES)

The amino acids at the tES dimerization intersection were identified for nucleotide substitution based on interatomic distance using the PyMOL software. The PRSF plasmid carrying tES gene19 was purified using OMEGA E.Z.N.A plasmid DNA mini kit I (D6922-02). The primers for nucleotide replacement were designed using QuikChange Primer Design and the PCR reaction performed using QuikChange lightening Site-Directed Mutagenesis Kit (210519). The PCR product was transformed into competent cells provided by the kit. The colonies were selected using Kanamycin (25 μg·mL-1; ThermoFisher, Singapore) resistance LB agar (Axil Scientific, USA) plates and subsequently sequenced for positive confirmation. The positive constructs were transformed into BL21 (DE3) E. coli competent cells. An overnight starter culture (25 ml) prepared from a single positive colony was used to inoculate LB broth (1000 mL) maintained at 37° C. and supplemented with Kanamycin. The bacteria culture was grown until the absorbance (OD600) of ˜0.7 was obtained. Protein expression was induced using isopropyl β-D-1-thiogalactopyranoside (IPTG, 0.4 mM; Axil Scientific), and the culture was allowed to grow for another 5 h at 37° C. The bacteria were pelleted by centrifugation at 14,000×g for 15 min. The pellet was resuspended in the lysis buffer (50 mM Tris-HCl, pH 8.0, 200 mM NaCl, 5 mM BME, and 0.1% Triton-X 100) and incubated on ice for 15 min, followed by sonication and centrifugation. The supernatant obtained was purified using a two-step chromatography procedure. The lysed sample were subjected to hydrophobic interaction chromatography (HIC) using HiPrep™ Phenyl FF (low sub) 16/10 column (Cytivia), followed by size-exclusion chromatography (SEC) using a Superdex S-200 10/300 GL column (Cytivia). The purity of the proteins was evaluated using sodium dodecyl sulfate-polyacrylamide gel electrophoresis (SDS-PAGE) and stored under reducing conditions for long-term storage.

### Characterization of DS-tES

The particle sizes of tES and DS-tES were measured by dynamic light scattering (DLS) studies (Nanobrook Omni, Brookhaven). The purified tES and DS-tES were aliquoted in sized cuvettes and the measurements were achieved at a scattering angle of 90°. The formation of disulfide bonds was assessed using non-reducing SDS-PAGE. Also, the concentration of free thiols present in the DS-tES was determined using Ellman's Assay. Briefly, 5,5′-dithio-bis-2-(nitrobenzoic acid) or DTNB prepared at 4 mg/ml concentration in 0.1 M Sodium Phosphate buffer was mixed with protein samples and the generation of the yellow adduct was measured at 412 nm. A standard graph was determined using known concentrations of L-cysteine and the free thiols were quantified. Additionally, the presence of endotoxin in the purified protein samples was determined using an endotoxin kit (Thermo Fisher Scientific, Waltham, MA, USA) as per the user instructions.

### Preparation of DS-tES-HRP/rluc

The DS-tES in presence of the reducing agent was acidified to pH 5.8 to obtain the subunits. The subunits were mixed with a 10-fold molar excess of HRP (Thermo Fisher Scientific, Waltham, MA, USA), and incubated at 4° C. for 30 minutes. The pH of the mixture was increased to 8.0 and the assembled DS-tES encapsulating HRP (DS-tES-HRP) was separated from free HRP using a non-reducing buffer on SEC. The HRP encapsulation was analyzed using SDS-PAGE.

### In Vitro Experiments

### Pepsin Assay

DS-tES-HRP/tES-HRP/HRP were incubated for 2h in the presence of 1 mg/ml pepsin. The molar ratio of HRP to Pepsin used in this study was 1:100 (for DS-tES-HRP and tES-HRP the equivalent HRP activity as free HRP was used) and 3 M HCl was used to make different pH values ranging from 2 to 8 and pH was reverted to 8 by NaOH to deactivate the Pepsin enzyme. The HRP activity was assayed after 2h using 3,3′,5,5′-tetramethylbenzidine (TMB) substrate in a 96-well crystal-clear polystyrene plate (Greiner Bio-One). Absorbance was measured at 450 nm using a Perkin Plate reader.

### Trypsin Assay

DS-tES-HRP/tES-HRP/HRP were incubated for 2h in different concentrations of Trypsin (0.25 and 0.4%). The HRP activity was measured after 2h via 3,3′,5,5′-tetramethylbenzidine (TMB) substrate in a 96-well crystal-clear polystyrene plate (Griener Bio-one).

### Cell Permeability

The penetrance of the DS-tES into the epithelium of the intestine was carried out in the human colon carcinoma cell line, Caco-2. Briefly, 0.5 million Caco2 cells were seeded on transwell plates and incubated in 10% CO₂ incubator for 21 days at 37° C. Upon changing the media to HBSS, the DS-tES-HRP was added to the apical layer. The plate was incubated at 37° C. for 3 h and the media from basolateral area was assessed for HRP activity.

### In Vivo Experiments

To investigate the stability of DS-tES in vivo, 0.8 mM of DS-tES-rluc or equivalent activity of free rluc were orally administered to Balb/c mice. Followed by intraperitoneal injection of ViviRen™ In vivo Substrate (0.4 mM) in 3 and 24 h. The distribution of the bioluminescence was evaluated using IVIS Spectrum. In addition, mice faeces were collected to assess the presence of the enzyme in the gastrointestinal tract after 5, 24, 48, 72 h, and 1 week of oral administration of DS-tES-rluc.

### Example 2

### Design and Screening for Disulfide tES

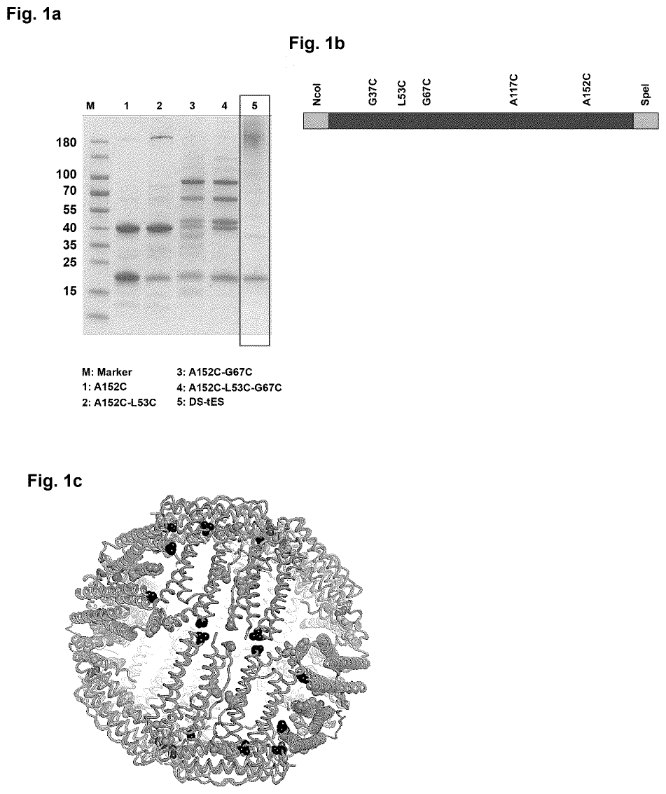

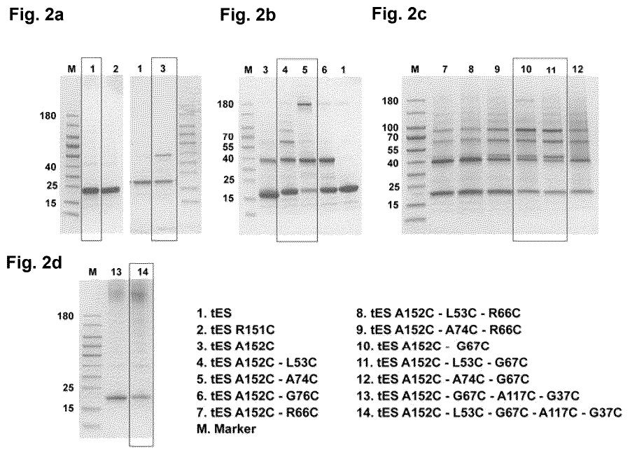

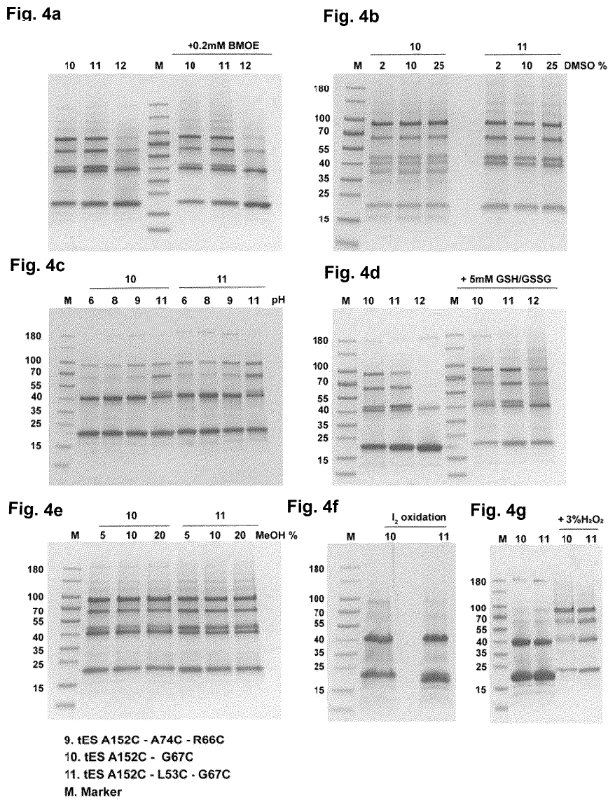

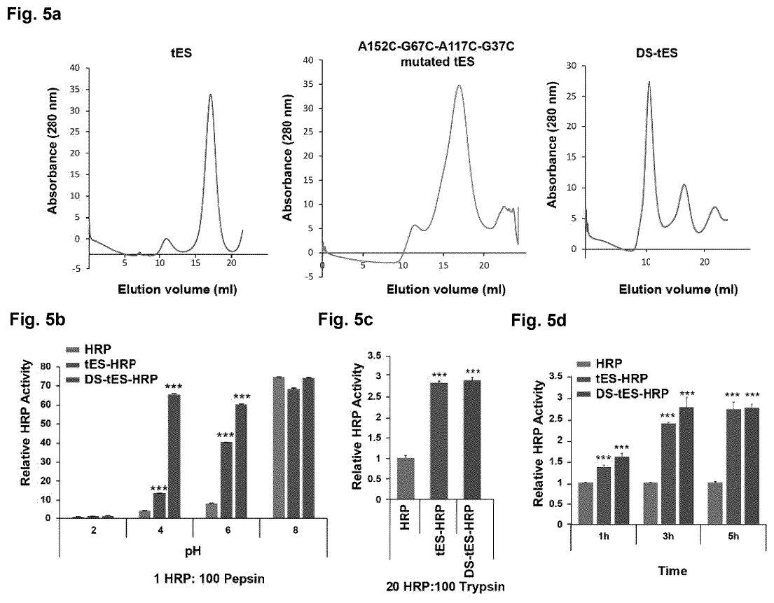

Previously, we reported the application of tES in delivering an iron-mediated catalytic center for intratumoral bioorthogonal catalysis and regression of solid tumors [Sadeghi, S., et al., ACS nano 16:10292-10301 (2022)]. To extend the potential applications for tES to additional delivery routes, we improved the stability of tES by introducing engineered disulfide bonds at subunit interfaces. The tES subunit amino acid sequence was first screened to find residues with ˜2 Å interatomic distance. We found that arginine 151(R151), alanine 152(A152), leucine 53(L53), alanine 74(A74), glycine 76(G76), arginine 66(R66), glycine 67(G67), glutamine 50(Q50), glycine 37(G37), and alanine 117(A117) are located in the tES dimerization intersection and thus designed mutagenesis primers to systematically substitute them to cysteine. As observed in non-reducing SDS-PAGE, R151C did not form disulfide bonds between the subunits whereas A152Cformed dimers (FIG. 2a). Subsequent double mutants (A152C-L53C and A152C-A74C) exhibited a higher propensity for disulfide bond formation between the subunits in comparison to A152C-G76C (FIG. 2b). Hence, the two variants were selected for further mutagenesis to form the double/triple mutants (A152C-R66C; A152C-G67C; A152C-L53C-R66C; A152C-A74C-R66C; A152C-L53C-G67C; and A152C-A74C-G67C) (FIG. 1a and FIG. 2c). Of these, A152C-G67C and A152C-L53C-G67C improved the stability on the non-reducing SDS-PAGE though the full linked shell was not observed (FIG. 2c). Diverse disulfide induction methodologies such as the use of dimethyl sulphoxide, glutathione system (GSH-GSSG) [Majoul, I., et al., FEBS Letters 401:104-108 (1997)], methanol, hydrogen peroxide, iodine [Montemiglio, L. C., et al., Nature Communications 10:1-8 (2019)], BMOE cross linker, and air oxidation in diverse pH were adopted. However, none of these resulted in the formation of fully linked and stabilized tES (FIGS. 4a-4g). Further screening revealed the significance of G37C and A117C in stabilizing tES in a non-reducing environment (FIG. 1a-1c and FIG. 2d). Therefore, the A152C-G67C and A152C-L53C-G67C tES were mutated to their corresponding constructs, A152C-G67C-A117C-G37C and A152C-L53C-G67C-A117C-G37C, and evaluated for their stability at pH 4.0. Interestingly, A152C-L53C-G67C-A117C-G37C (DS-tES) exhibited higher stability at the acidic pH and was selected for further studies (FIG. 5a). All the clones were sequence confirmed, mutants expressed, and proteins purified and evaluated in a non-reducing SDS-PAGE.

### Example 3

### Purification and Characterization of DS-tES

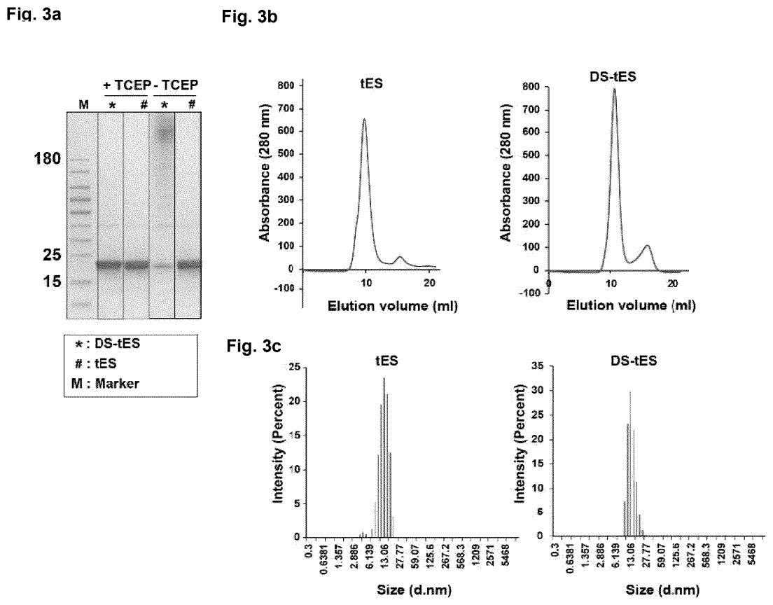

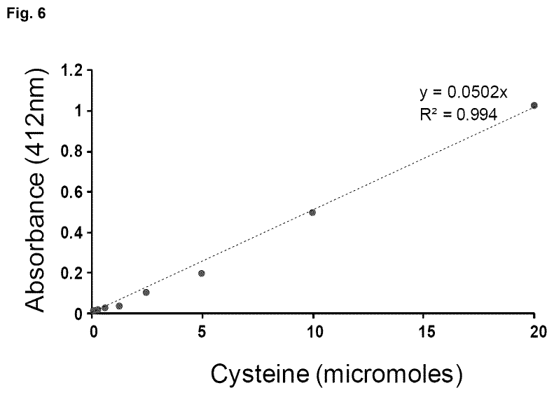

As indicated in FIGS. 1a and 1b, mutagenesis at the five target sites (A152C-L53C-G67C-A117C-G37C) contributed to the DS-tES with a hydrodynamic diameter of ˜13 nm and elution of 10 ml in size exclusion chromatography (FIGS. 3b and 3c). Also, reduction with TCEP to single subunits suggested that DS-tES was more stable than tES in a non-reducing SDS-PAGE environment where it remained largely intact even in the presence of denaturants (FIG. 2 and FIG. 3a). Compared to tES, which dissociates into monomers at pH 4.0, DS-tES remained fully assembled and folded in extremely acidic pH (FIG. 5a). Thiolation is an efficient method for customization of the mucoadhesive and mechanical properties of drug delivery systems, therefore the thiol group content in the DS-tES was quantified using Ellman's reagent and it was determined to be 1.2 micromoles per mg of protein (FIG. 6). Further, the endotoxin levels in the purified protein samples were estimated to be 0.018 ng/ml per mg of protein and were within the limits reported previously. Overall, the cage was found to be covalently ‘locked’ and adopted an extremophile stability profile. This new quality is the result of 60 rationally engineered disulfide bonds that stabilize inter-subunit interactions without affecting the overall morphology of tES. A comparison of the characteristics of DS-TES and tES is shown in Table 2.

TABLE 2
Comparative analysis of DS-tES and tES

| Features | DS-tES | tES |
|---|---|---|
| Number of cysteine per | 5 | 0 |
| subunit | | |
| Non-reducing SDS- | Majorly oxidized Shell | Subunits |
| PAGE | | |
| Molecular diameter | ~13 nm | ~13 nm |
| Stable at acidic pH (~4) | Yes | No |
| Against pepsin digestion | Highly stable | Not stable |
| at acidic pH | | |
| Permeable through | High | High |
| Caco2 monolayer | | |

### Example 4

### DS-tES Stabilized HRP Activity in Simulated GI Conditions

Acidic gastric fluid, together with enzymatic barriers such as pepsin and trypsin, is biologically designed to degrade proteins and thus prohibits the administration of most protein-based therapeutics. The stability of HRP as a model peptide encapsulated within the DS-tES (DS-tES-HRP) was thus evaluated in a simulated GI environment, and the activity was compared to free HRP or tES-HRP (non-disulfide stabilized). In the presence of pepsin and pH 4.0, DS-tES-HRP exhibited ˜5 times and ˜14 times more enzymatic activity compared to tES-HRP or free HRP, respectively (FIG. 5b). However, when incubated with trypsin at pH 7.0, both DS-tES and tES protected the encapsulated HRP against proteolysis equally (FIG. 5c). The protective property of tES towards the encapsulated protein cargo from trypsinolysis was previously reported [Deshpande, S. et al., Nature Communications 8:1-8 (2017)]. Similarly, tES maintained ˜30% of HRP activity when incubated with serum proteases for a duration of 8-days, but with no quantifiable activity observed with free HRP [Sadeghi, S., et al., ACS nano 16:10292-10301 (2022)]. Thus, DS-tES protects internalized protein substrate from the two major GI proteases in both normal and acidic pH environments.

### Example 5

### DS-tES Permeabilizes Through Intestinal Cell Layers

To assess transcytosis of DS-tES through intestinal epithelium, a permeability assay was carried out in human colon carcinoma cell line Caco2 as it represents the best available in vitro model of small intestine enterocytes. As evident from FIG. 5d, the HRP signal was significantly higher upon encapsulation in DS-tES at all points evaluated. DS-tES-HRP exhibited 3 times more peroxidase activity in comparison to free HRP after 3 hours. Also, a mild reduction in peroxidase activity from DS-tES to tES was observed wherein the difference could be attributed to the free thiols present on the DS-tES. Similar thiol-mediated permeabilization through the Caco2 monolayers was reported previously [Zhang, M., et al., International Journal of Biological Macromolecules 138:636-647 (2019); Martien, R., et al., Nanotechnology 19: 045101 (2008)]. Taken together, these observations suggest a promising role for DS-tES in intestinal epithelial transfer.

### Example 6

### Oral Delivery of DS-tES

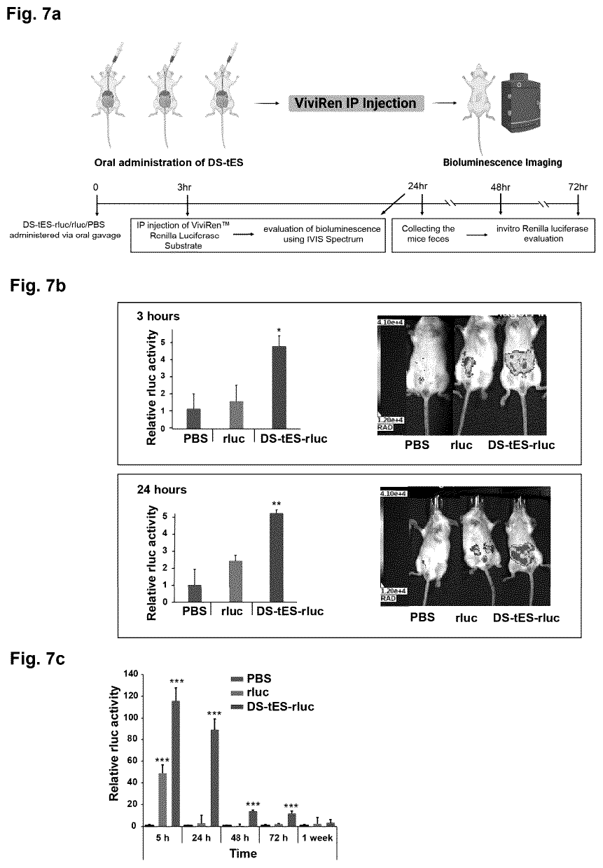

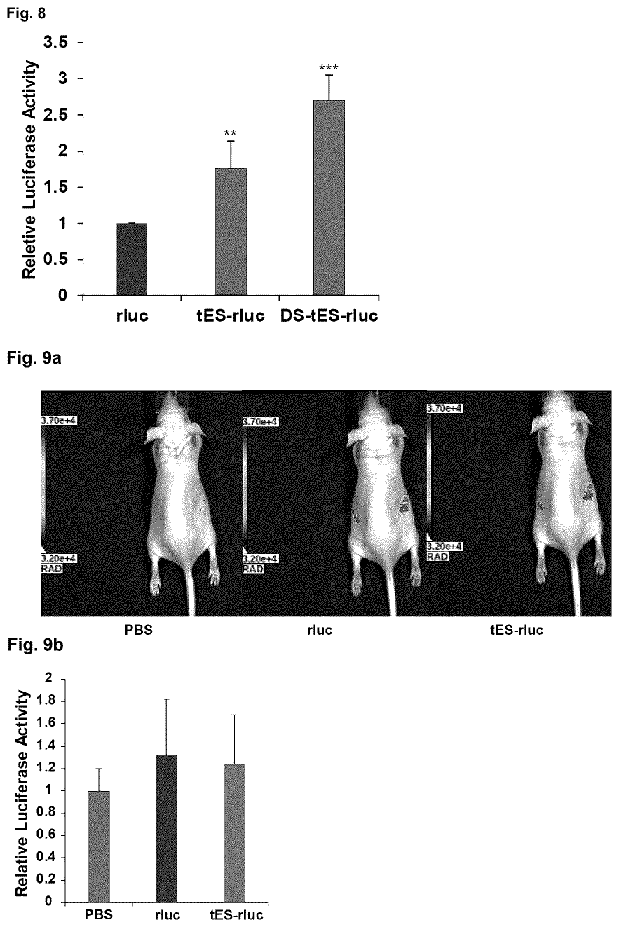

To further understand the in vivo capabilities of DS-tES as an oral delivery vehicle, a single dose of both rluc and rluc encapsulated within DS-tES (DS-tES-rluc) was orally administered to Balb/c mice. The Viviren substrate was then intraperitoneally injected into mice after 3 and 24 h (FIG. 7a). As can be seen in FIG. 7b, both rluc and DS-tES-rluc moved through the GI tract by 3 h post-administration. Over the next 24 h, the encapsulated luciferase activity was maintained in the GI tract but negligible activity was observed with free rluc. Thereafter, the total signal decreased over time as rluc, whether free or encapsulated, was eliminated from the GI tract. Additionally, the rluc activity was measured from mice faeces. A strongly significant effect of DS-tES (P<0.001) was observed which protected rluc activity for the length of the GI tract up to 24 h. The luciferase activity was then reduced ˜6 times from 24 h to 72 h. On the contrary, the free luciferase activity was reduced by more than 50 times after 24 h, suggesting that DS-tES could protect and maintain the luciferase activity upon oral administration (FIG. 7c).

### SUMMARY

The transit of protein nanoparticles through the GI tract involves exposure to a high pH gradient (pH 2.0-8.0), digestive enzymes (pepsin, trypsin, etc.), and mucosal and cellular barriers, all of which alter the structure-function of the loaded therapeutic proteins or peptides. The present invention illustrates that disulfide-linked tES (DS-tES) protects encapsulated substrates from such an adverse gastro-intestinal (GI) environment. The high abundance of cysteines and disulfide bonds, which stabilize the quaternary structure, is one of the most notable structural properties of DS-tES. The free thiols may have an additional role in cellular absorption, specifically through transferrin receptor mediated endocytosis. We also noticed that when taken orally, DS-tES encapsulating Renilla luciferase had a long residence period within the GI tract before being removed from the system. As DS-tES utilize a simple and rapid encapsulation technique without affecting the properties of the encapsulated substrate, it may have significant implications for the nutraceutical and pharmaceutical industries.

### REFERENCES

1. Sadeghi, S., et al. Bioorthogonal Catalysis for Treatment of Solid Tumors Using Thermostable, Self-Assembling, Single Enzyme Nanoparticles and Natural Product Conversion with Indole-3-acetic Acid. ACS nano 16:10292-10301 (2022).
2. Majoul, I., Ferrari, D. & Söling, H.-D. Reduction of protein disulfide bonds in an oxidizing environment: The disulfide bridge of cholera toxin A-subunit is reduced in the endoplasmic reticulum. FEBS letters 401, 104-108 (1997).
3. Okumura, M., Shimamoto, S. & Hidaka, Y. A chemical method for investigating disulfide-coupled peptide and protein folding. The FEBS Journal 279, 2283-2295 (2012).
4. Deshpande, S., et al. Thermostable exoshells fold and stabilize recombinant proteins. Nature communications 8, 1-8 (2017).
5. Zhang, M., et al. The enhancing effect of N-acetylcysteine modified hyaluronic acid-octadecylamine micelles on the oral absorption of paclitaxel. International journal of biological macromolecules 138, 636-647 (2019).
6. Martien, R., Loretz, B., Sandbichler, A. M. & Schnuerch, A. B. Thiolated chitosan nanoparticles: transfection study in the Caco-2 differentiated cell culture. Nanotechnology 19, 045101 (2008).
7. Wu, W., Kong, X., Zhang, C., Hua, Y. & Chen, Y. Improving the stability of wheat gliadin nanoparticles—Effect of gum arabic addition. Food Hydrocolloids 80, 78-87 (2018).
8. Ezpeleta, I., et al. Preparation of Ulex europaeus lectin-gliadin nanoparticle conjugates and their interaction with gastrointestinal mucus. International journal of pharmaceutics 191, 25-32 (1999).
9. Bies, C., Lehr, C.-M. & Woodley, J. F. Lectin-mediated drug targeting: history and applications. Advanced drug delivery reviews 56, 425-435 (2004).
10. Minko, T. Drug targeting to the colon with lectins and neoglycoconjugates. Advanced drug delivery reviews 56, 491-509 (2004).
11. Penalva, R., et al. Casein nanoparticles as carriers for the oral delivery of folic acid. Food hydrocolloids 44, 399-406 (2015).
12. Shapira, A., Assaraf, Y. G. & Livney, Y. D. Beta-casein nanovehicles for oral delivery of chemotherapeutic drugs. Nanomedicine: Nanotechnology, Biology and Medicine 6, 119-126 (2010).
13. Penalva, R., et al. Zein-based nanoparticles improve the oral bioavailability of resveratrol and its anti-inflammatory effects in a mouse model of endotoxic shock. Journal of agricultural and food chemistry 63, 5603-5611 (2015).
14. Umamaheshwari, R., Ramteke, S. & Jain, N. K. Anti-Helicobacter pylori effect of mucoadhesive nanoparticles bearing amoxicillin in experimental gerbils model. Aaps Pharmscitech 5, 60-68 (2004).
15. Irache, J. M. & González-Navarro, C. J. Zein nanoparticles as vehicles for oral delivery purposes. Vol. 12 1209-1211 (Future Medicine, 2017).
16. Patel, A. R., Bouwens, E. C. & Velikov, K. P. Sodium caseinate stabilized zein colloidal particles. Journal of Agricultural and Food Chemistry 58, 12497-12503 (2010).
17. Hong, S., et al. Protein-based nanoparticles as drug delivery systems. Pharmaceutics 12, 604 (2020).
18. Kianfar, E. Protein nanoparticles in drug delivery: animal protein, plant proteins and protein cages, albumin nanoparticles. Journal of Nanobiotechnology 19, 1-32 (2021).
19. Vallerinteavide Mavelli, G., Sadeghi, S., Vaidya, S. S., Kong, S. N. & Drum, C. L. Nanoencapsulation as a General Solution for Lyophilization of Labile Substrates. Pharmaceutics 13, 1790 (2021).
20. Estrada, L. H. & Champion, J. Protein nanoparticles for therapeutic protein delivery. Biomaterials science 3, 787-799 (2015).
21. He, J., Fan, K. & Yan, X. Ferritin drug carrier (FDC) for tumor targeting therapy. Journal of Controlled Release 311, 288-300 (2019).
22. Khoshnejad, M., Parhiz, H., Shuvaev, V. V., Dmochowski, I. J. & Muzykantov, V. R. Ferritin-based drug delivery systems: Hybrid nanocarriers for vascular immunotargeting. Journal of Controlled Release 282, 13-24 (2018).
23. Sadeghi, S., et al. A general approach to protein folding using thermostable exoshells. Nature communications 12, 1-15 (2021).
24. Yin, S., Davey, K., Dai, S., Liu, Y. & Bi, J. A critical review of ferritin as a drug nanocarrier: Structure, properties, comparative advantages and challenges. Particuology 64, 65-84 (2022).
25. Landreh, M., et al. Predicting the shapes of protein complexes through collision cross section measurements and database searches. Analytical Chemistry 92, 12297-12303 (2020).
26. Deng, J., et al. A review of food-drug interactions on oral drug absorption. Drugs 77, 1833-1855 (2017).
27. Dressman, J. B., et al. Upper gastrointestinal (GI) pH in young, healthy men and women. Pharmaceutical research 7, 756-761 (1990).
28. Russell-Jones, G. The potential use of receptor-mediated endocytosis for oral drug delivery. Advanced drug delivery reviews 20, 83-97 (1996).
29. Li, H., Sun, H. & Qian, Z. M. The role of the transferrin-transferrin-receptor system in drug delivery and targeting. Trends in pharmacological sciences 23, 206-209 (2002).
30. Koneru, T., et al. Transferrin: biology and use in receptor-targeted nanotherapy of gliomas. ACS omega 6, 8727-8733 (2021).
31. Candelaria, P. V., Leoh, L. S., Penichet, M. L. & Daniels-Wells, T. R. Antibodies targeting the transferrin receptor 1 (TfR1) as direct anti-cancer agents. Frontiers in immunology 12, 583 (2021).
32. Tortorella, S. & C Karagiannis, T. The significance of transferrin receptors in oncology: the development of functional nano-based drug delivery systems. Current drug delivery 11, 427-443 (2014).
33. Montemiglio, L. C., et al. Cryo-EM structure of the human ferritin-transferrin receptor 1 complex. Nature communications 10, 1-8 (2019).
34. Shen, Y., et al. Transferrin receptor 1 in cancer: a new sight for cancer therapy. American journal of cancer research 8, 916 (2018).
35. Lodhi, M. S., et al. Probing Transferrin Receptor Overexpression in Gastric Cancer Mice Models. ACS omega 6, 29893-29904 (2021).
36. Banerjee, D., Flanagan, P. R., Cluett, J. & Valberg, L. S. Transferrin receptors in the human gastrointestinal tract: relationship to body iron stores. Gastroenterology 91, 861-869 (1986).
37. Widera, A., Bai, Y. & Shen, W.-C. The transepithelial transport of a G-CSF-transferrin conjugate in Caco-2 cells and its myelopoietic effect in BDF1 mice. Pharmaceutical research 21, 278-284 (2004).
38. Yong, J. M., Mantaj, J., Cheng, Y. & Vllasaliu, D. Delivery of nanoparticles across the intestinal epithelium via the transferrin transport pathway. Pharmaceutics 11, 298 (2019).
39. Du, W., et al. Transferrin receptor specific nanocarriers conjugated with functional 7peptide for oral drug delivery. Biomaterials 34, 794-806 (2013).
40. Liang, M., et al. H-ferritin-nanocaged doxorubicin nanoparticles specifically target and kill tumors with a single-dose injection. Proceedings of the National Academy of Sciences 111, 14900-14905 (2014).
41. Calisti, L., et al. Engineered ferritin for lanthanide binding. PloS one 13, e0201859 (2018).
42. Laurent, Q., et al. Thiol-Mediated Uptake. Jacs Au 1, 710-728 (2021).
43. Cheng, Y., et al. Inhibitors of thiol-mediated uptake. Chemical science 12, 626-631 (2021).
44. Puri, V., Sharma, A., Kumar, P. & Singh, I. Thiolation of biopolymers for developing drug delivery systems with enhanced mechanical and mucoadhesive properties: A review. Polymers 12, 1803 (2020).

1. An engineered disulphide-linked ferritin assembly comprising at least one modified ferritin subunit, wherein the at least one modified ferritin subunit comprises the amino acid sequence set forth in SEQ ID NO: 1, and comprises;
i) a F116H substitution, and zero or more amino acid substitutions at one or more positions selected from the group comprising E65, E128, E131, and D138 of SEQ ID NO: 1, and
ii) a Cys substitution at two or more positions selected from the group comprising G37, L53, R66, G67, A74, A117 and A152 of SEQ ID NO: 1.

2. The engineered disulphide-linked ferritin assembly of claim 1, wherein the assembly possesses a net negative interior charge, net positive interior charge, or a net neutral interior charge.

3. The engineered disulphide-linked ferritin assembly of claim 2, wherein the modified ferritin subunit comprises an amino acid substitution at any one or more positions selected from E65, E128, E131, and D138.

4. The engineered disulphide-linked ferritin assembly of claim 3, wherein the amino acid substitution comprises;
i) substitutions E65K, E128K, E131K and D138A, which cause the assembly to have a net positive interior charge; or
ii) substitutions E65Q and D138A, which cause the assembly to have a net neutral interior charge.

5. The engineered disulphide-linked ferritin assembly of claim 1, wherein prior to Cys substitution the modified ferritin subunit comprises an amino acid sequence selected from the group comprising SEQ ID NO: 2, SEQ ID NO: 3, and SEQ ID NO: 4.

6. The engineered disulphide-linked ferritin assembly of claim 1, wherein the modified ferritin subunit comprises cysteine residues at positions selected from the group comprising A152C-G67C-A117C-G37C and A152C-L53C-G67C-A117C-G37C.

7. The engineered disulphide-linked ferritin assembly of claim 6, wherein the modified ferritin subunit comprises an amino acid sequence selected from the group comprising SEQ ID NO: 5, SEQ ID NO: 6, SEQ ID NO: 7, SEQ ID NO: 8, SEQ ID NO: 9, and SEQ ID NO: 10.

8. The engineered disulphide-linked ferritin assembly of claim 1, wherein a bioorthogonal catalytic centre, polypeptide, or nucleic acid, or any combination thereof, is encapsulated within the ferritin assembly.

9. The engineered disulphide-linked ferritin assembly of claim 1, wherein the disulphide-linked ferritin assembly further comprises a cell-targeting moiety.

10. An in vitro method of forming an engineered disulphide-linked ferritin assembly, the method comprising adjusting the pH from an acidic pH to basic pH of a sample comprising at least one modified ferritin subunit of claim 1, wherein the assembly is stable in the gastrointestinal tract and capable of oral drug delivery.

11. The method of claim 10, further comprising a bioorthogonal catalytic centre, polypeptide or a nucleic acid, or any combination thereof, in the sample whereby said bioorthogonal catalytic centre, polypeptide or nucleic acid, or any combination thereof, is encapsulated by the engineered ferritin assembly at basic pH of the sample, wherein the acidic pH in the sample is at least about 4.0 and/or the basic pH in the sample is about 8.0.

12. (canceled)

13. A method of delivering an engineered disulphide-linked ferritin assembly into a cell, comprising contacting the cell with a disulphide-linked ferritin assembly of claim 1.

14. An isolated plasmid or vector polynucleic acid comprising a sequence that encodes a modified ferritin subunit, wherein the modified ferritin subunit is as defined in claim 1.

15. A composition comprising at least one engineered disulphide-linked ferritin assembly of claim 1 for use in the prophylaxis or treatment of disease in a subject, wherein the at least one engineered disulphide-linked ferritin assembly has encapsulated:
i) an enzyme; or
ii) an enzyme and one or more additional therapeutic agents.

16. (canceled)

17. The composition of claim 14, which is capable of drug delivery by the oral route.

18. A method of treatment or prophylaxis comprising administering to a subject in need of such treatment or prophylaxis an efficacious amount of a composition of claim 14.

19. The method of claim 16, wherein the composition is administered sequentially or simultaneously with another therapeutic agent.

20. The method of claim 17, wherein the composition comprises a bioorthogonal catalytic centre encapsulated within the engineered disulphide-linked ferritin assembly and the other therapeutic agent is a substrate for said catalytic centre.

21. Use of a composition of claim 14 for the manufacture of a medicament for the treatment of a disease in a subject.

22. (canceled)

23. A kit, to encapsulate a bioorthogonal catalytic centre, polypeptide, or nucleic acid, or any combination thereof, within an engineered disulphide-linked ferritin assembly, the kit comprising at least one modified ferritin subunit according to claim 1, and/or the plasmid or vector nucleic acid.

24. (canceled)
